(19) United States
(12) Patent Application Publication
(10) Pub. No.: US 2024/0360079 A1
(43) Pub. Date: Oct. 31, 2024

(54) PALLADIUM FREE PROCESSES FOR PREPARATION OF ACRYLATE COMPOUNDS

(71) Applicant: AMGEN INC., Thousand Oaks, CA (US)

(72) Inventors: Adrian ORTIZ, Oak Park, CA (US); Jaika DOERFLER, Glendale, CA (US); Karina R. VAIDA, Burlington, MA (US)

(73) Assignee: AMGEN INC., Thousand Oaks, CA (US)

(21) Appl. No.: 18/291,285

(22) PCT Filed: Aug. 17, 2022
(86) PCT No.: PCT/US22/40665

§ 371 Date: Jan. 23, 2024

Related U.S. Application Data
(60) Provisional application No. 63/234,927, filed on Aug. 19, 2021.

(51) Int. Cl. C07C 319/12; C07C 321/26; C12P 13/00

(52) U.S. Cl. CPC C07C 319/12; C07C 321/26; C12P 13/001

(57) ABSTRACT

The present invention relates to precious metal (Pd/Pt/Rh/Ru) free, preferably Pd free, processes for preparing a compound having formula (1) wherein: R¹, R², and X are as defined in the specification, or a salt thereof. Preferably, the compound is an iodo acrylate compound.

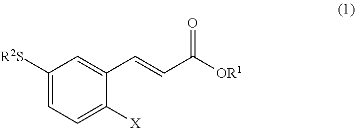

### FIELD OF THE INVENTIONS

The present invention relates to novel processes for palladium free preparation of acrylate compounds, including iodo acrylate compounds, having a high stereomeric ratio.

### BACKGROUND OF THE INVENTION

The present application relates to precious metal, including Pd, Pt, Rh, or Ru free, preferably Pd free, processes for preparing a compound having formula (1):

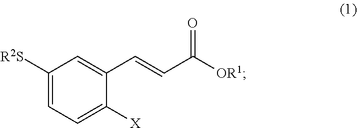

wherein: R¹, R², and X are as defined below, or a salt thereof. Preferably, the compound is an iodo acrylate compound, including ethyl (E)-3-(5-(benzylthio)-2-iodophenyl) acrylate (Compound 1a).

In the known route, compound (1) was prepared from starting material compound (2). Specifically, compound (1a), which is compound (1) wherein R¹ is ethyl; R² is benzyl; and X is I; was prepared from a starting material aniline acrylate compound (2a), which is compound (2) wherein R¹ is ethyl and R² is benzyl:

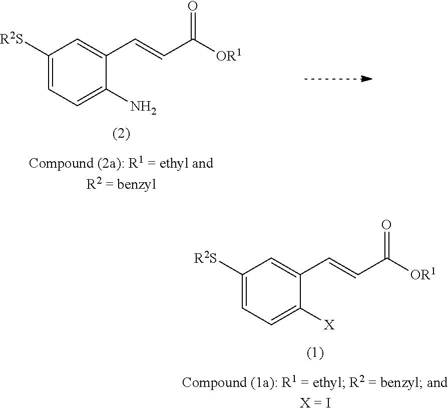

Current preparation of compound (2a) employs back to back palladium (Pd) mediated cross couplings reactions as follows:

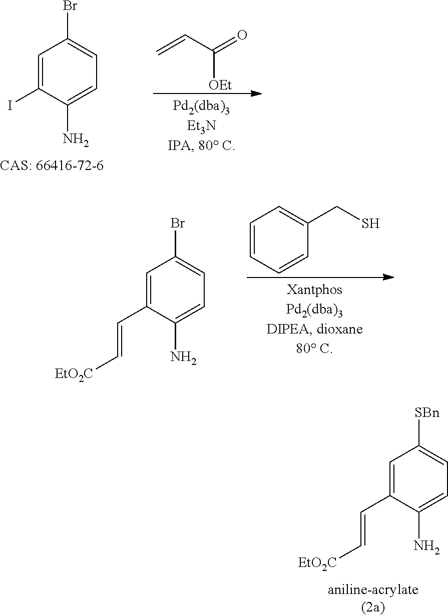

Greater than 50% of the cost of goods manufacturing (COGM) is derived from the use of Pd. As a result, despite rapid synthesis, this method of preparation of compound (2a) is very expensive at about US$8,000/Kg.

Furthermore, the use of back-to-back Pd transformations leads to robustness challenges, such as the need for high Pd loading (6 mol % and 5 mol %), and difficulties in Pd-removal downstream.

In order to drive cost down and to improve overall process robustness, there is a need to develop an improved novel synthetic technology to prepare compound (2a) which will serve as a key intermediate compound to prepare compound (1a) or analogs thereof.

The present inventors have developed a novel synthetic route which eliminates all use of expensive precious metals, including Pd, to address these cost and robustness challenges. In the improved process of preparation of compound (2) and (2a) of the invention, the amino group is prepared through a reduction of a nitro group with Iron, an inexpensive base metal. The present inventors have further developed an alternative novel bio-catalytic nitroreduction with nitroreductase enzyme, which improved the yield of compound (2a).

### SUMMARY OF THE INVENTION

The present invention provides a precious metal catalyst free, preferably palladium metal catalyst free, improved, safer, cost-effective and easy to operate on plant scale process for synthesis of acrylate compounds, including halo acrylate compounds.

In embodiment 1, the present invention provides a process for the preparation of a compound having formula (1):

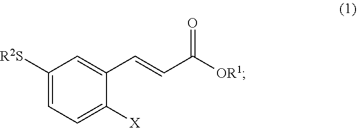

or a salt thereof:

wherein:

X is halo, CN, CF₃, or OH;

R¹ is (C₁-C₆) alkyl; a 5-, 6-, 7-, 8-, 9-, or 10-membered aryl or heteroaryl; or a 3-, 4-, 5-, 6-, 7-, 8-, 9-, or 10-membered cycloalkyl or heterocycloalkyl group;

wherein the heteroaryl or heterocycloalkyl group can have from 1 to 3 heteroatoms independently selected from O, N or S;

or a carbon atom in the cycloalkyl or heterocycloalkyl group can be part of a C═O group;

R² is selected from (C₁-C₆) alkyl or benzyl; comprising:

(a) contacting a compound of formula (2):

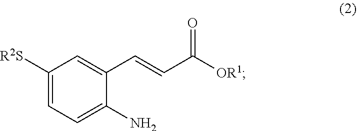

wherein said R¹ and R² are as defined above in compound (1); with an acid HA in a solvent-1, and in the presence of a nitrous salt;

(b) introducing a halogenating agent (including fluorinating, chlorinating, brominating, or iodinating agent), a cyanating agent, a trifluoromethylation agent, or a hydroxylating agent, optionally in the presence of a solvent-2, to form said compound (1); wherein said solvent-1 and solvent-2 can be identical or different; and

Wherein said compound (2) is prepared in a process free of palladium catalyst.

In a sub-embodiment of embodiment 1, in (a), said acid HA is HCl, HBr, HI, p-TsOH, or H₂SO₄. Preferably the acid HA is HCl.

In a further sub-embodiment of embodiment 1, in (a), said solvent-1 is water, THF, methyl THF, CH₃CN, or (C₁-C₆)alkyl acetate solvent, or mixtures thereof. Preferably, said solvent-1 is ethyl acetate or isopropyl acetate.

In a further sub-embodiment of embodiment 1, in (a), said nitrous salt is NaNO₂.

In a further sub-embodiment of embodiment 1, in (a), said low temperature is between −10° C. to 10° C.; or between 0° C. to 5° C. Preferably, between 0° C. to 5° C.

In a further sub-embodiment of embodiment 1, in (b), said halogenating agent is a metal halide salt; said cyanating agent is CuCN; said trifluoromethylation agent is CuCF₃; and said hydroxylating agent is Cu₂O/Cu(II). Preferably, in (b), a halogenating agent is used, more preferably said halogenating agent is a KI or CuI.

In a further sub-embodiment of embodiment 1, in (b), said solvent-2 is water, THF, methyl THF, CH₃CN, or (C₁-C₆)alkyl acetate solvent, or mixtures thereof. Preferably, in (b), said solvent-2 is (C₁-C₆)alkyl; more preferably, isopropyl acetate.

In embodiment 2, the present invention provides a process according to embodiment 1, further comprising preparing said compound (2) comprising:

(c1) activating a metal in the presence of an acidic salt HA¹ in a polar solvent; and contacting a compound of formula (3):

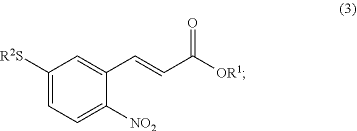

wherein said R¹ and R² are as defined above in compound (1):

with said activated metal at an elevated temperature in a polar solvent to form said compound (2) or a salt thereof:

or alternatively, the process further comprising preparing said compound (2) comprising:

(c2) reacting said compound of formula (3), optionally in the presence of a co-solvent, with an enzymatic reducing agent in an aqueous buffer solution, and in the presence of at least one catalyst and a co-factor, to form said compound of formula (2), or a salt thereof.

In embodiment 3, the present invention provides a process according to embodiment 2, further comprising preparing said compound (2) comprising:

(c1) activating a metal in the presence of an acidic salt HA¹ in a polar solvent; and contacting said compound of formula (3) with said activated metal at an elevated temperature in a polar solvent to form said compound (2) or a salt thereof.

In embodiment 4, the present invention provides a process according to embodiment 2, further comprising preparing compound (2) comprising:

(c2) contacting said compound of formula (3), in the presence of a co-solvent, with an enzymatic reducing agent in an aqueous buffer, and in the presence of a metal catalyst, a co-catalyst, and a co-factor, to form said compound of formula (2), or a salt thereof.

In a sub-embodiment of any of embodiments 2 or 3, in (c1), said metal is selected from Fe°, Zn°, Pd°, Pt°, Ru°, or Rh°. Preferably, said metal is Fe°.

In a further sub-embodiment of any of embodiments 2 or 3, in (cl), said acidic salt HA1 is ammonium chloride, acetic acid, or HCl. Preferably, said acidic salt HA' is ammonium chloride.

In a further sub-embodiment of any of embodiments 2 or 3, in (c1), said polar solvent is water, (C1-C6) alkyl alcohol, or mixture thereof. Preferably, said polar solvent is water and ethanol mixture.

In a further sub-embodiment of any of embodiments 2 or 3, in (c1), said elevated temperature is between 50° C. to 90° C.; or 75° C. to 80° C. Preferably, said temperature is 75° C. to 80° C.

In a further sub-embodiment of any of embodiments 2 or 4, in (c2), said co-solvent is selected from DMSO, or water/DMSO mixture. Preferably, the co-solvent is 20 vol % to 30 vol % DMSO. More preferably, the co-solvent is 30 vol % DMSO.

In a further sub-embodiment of any of embodiments 2 or 4, in (c2), said buffer is selected from phosphate, PIPES, TRICINE, BICINE, HEPES, TRIS, TES, CAPS, Kpi, or CHES. Preferably, the buffer is TRICINE.

In a further sub-embodiment of any of embodiments 2 or 4, in (c2), said enzymatic reducing agent is a nitroreductase (NR) enzyme. Preferably the enzyme is NR-55.

In a further sub-embodiment of any of embodiments 2 or 4, in (c2), said catalyst is a metal catalyst and a co-catalyst: wherein said metal catalyst is a vanadium catalyst selected from V₂O₅, NH₃VO₄. V(IV) oxide phthalocyanine, V(IV) oxide bis (2,4-pentanedionate), vanadyl sulfate hydrate, V(V)oxy triethoxide, 3% V/C, or V(III)2,4-pentanedionate.

In a further sub-embodiment of any of embodiments 2 or 4, in (c2), said catalyst is a metal catalyst and a co-catalyst; wherein said metal catalyst is V₂O₅ or NH₃VO₄, and said co-catalyst is GDH-101 and sugar, and wherein said co-factor is NADP+. Preferably, the sugar is dextrose or glucose.

In a further sub-embodiment of any of embodiments 2 or 4, in (c2), said reaction is performed at between pH 7 or 8.

In a further sub-embodiment of any of embodiments 2 or 4, in (c2), said compound of formula (3), which is dissolved in said co-solvent, is slowly added to the enzymatic reducing agent in said aqueous buffer solution and in the presence of a metal catalyst, a co-catalyst, and co-factor. Preferably, said metal catalyst is vanadium metal, more preferably, V₂O₅ or NH₃VO₄. Preferably, said co-catalyst is GDH-101 and sugar. Preferably, the sugar is dextrose or glucose. Preferably, said co-factor is NADP+.

In a further sub-embodiment of any of embodiments 2 or 4, the product of (c2) is a salt selected from a halide salt, selected from HCl salt or HBr salt, or a sulphonic acid salt, selected from mesylate salt, tosylate salt, or aryl sulphonate salt. Preferably, the salt is a halide salt, more preferably HCl salt. A more preferred product of (c2) is

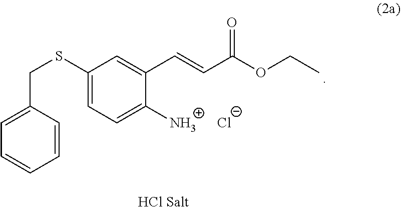 [SMILES: CCOC(=O)/C=C/c1cc(SCc2ccccc2)ccc1[NH3+] ; [Cl-]]

In a further sub-embodiment of any of embodiments 2 or 4, in (c2), more preferably, the reaction is performed at an elevated temperature. Preferably, the temperature ranges from 40° C. to 50° C. More preferably, between 43° C. to 47° C. Most preferably 45° C.

In embodiment 5, the present invention provides a process according to embodiment 2, further comprising preparing said compound (3) comprising:

(d) contacting a compound of formula (4):

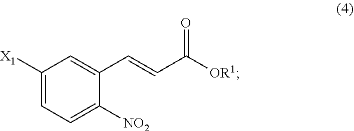

wherein X₁ is halo; and said R¹ is as defined above in compound (3);

with a thiol agent in the presence of a base, in an organic solvent, and at an elevated temperature: to form said compound (3), or a salt thereof.

In a sub-embodiment of embodiment 5, in (d), said X₁ in each of Compound (4) and (5) is fluoro or chloro. Preferably, X₁ is fluoro.

In a further sub-embodiment of embodiment 5, in (d), said base is a carbonate salt or phosphate salt. Preferably, said base is Cs₂CO₃ or K₃PO₄.

In a further sub-embodiment of embodiment 5, in (d), said organic solvent is selected from DMF, DMAc, or NMP.

In a further sub-embodiment of embodiment 5, in (d), said elevated temperature is between 50° C. to 85° C.; or between 65° C. to 80° C. Preferably said temperature is 70° C.

In a further sub-embodiment of embodiment 5, in (d), said thiol agent is C₆H₅CH₂SH or (C₁-C₆)alkyl-SH, such as CH₃SH. Preferably, said thiol agent is C₆H₅CH₂SH.

In a further sub-embodiment of embodiment 5, in (d), said reaction is performed under a low water content condition and no excess thiol agent is used to avoid any sulfur generation in the work up. Preferably, the water content concentration is kept below 1000 ppm. Preferably, the thiol agent is within 0.90 equivalent to 1.1 equivalent. In a preferred sub-embodiment, no sulfur was generated in the work up.

In embodiment 6, the present invention provides a process according to embodiment 5, further comprising preparation of said compound (4), or a salt thereof, comprising:

(e) contacting a compound of formula (5):

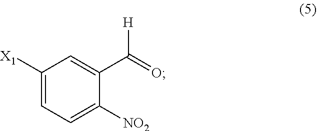

wherein X₁ is halo as defined in compound (4); with an alkenating agent, in the presence of a base, in an organic solvent to form said compound (4), or a salt thereof.

In a sub-embodiment of embodiment 6, in (e), said X₁ in each of Compound (4) and (5) is F or Cl.

In a further sub-embodiment of embodiment 6, in (e), said alkenylating agent is Wittig reagent (including triphenyl phosphonium ylide or ethyl 2-(diethoxyphosphoryl)acetate), or Horner-Wadsworth-Emmons (HWE) reagent. Preferably, the alkenylating agent is Wittig reagent. More preferably, ethyl 2-(diethoxyphosphoryl)acetate.

In a further sub-embodiment of embodiment 6, in (e), said organic solvent is selected from DIPEA, CH₃CN, TEA, N-methyl morpholine, or mixtures thereof. Preferably, the solvent is CH₃CN.

In a further sub-embodiment of embodiment 6, in (e), said halide salt is selected from LiCl or LiBr. Preferably the halide salt is LiCl.

In embodiment 7, the present invention provides a process according to any of the above embodiments 1, 2, 3, 4, 5, or 6, or any sub-embodiments thereof, wherein X in compound (1) is iodo.

In embodiment 8, the present invention provides a process according to any of the above embodiments 1, 2, 3, 4, 5, 6, or 7, or any sub-embodiments thereof, wherein X₁ in each of compounds (1) and (2) is fluoro or chloro.

In embodiment 9, the present invention provides a process according to any of the above embodiments 1, 2, 3, 4, 5, 6, 7, or 8, or any sub-embodiments thereof, wherein R¹ in each of compounds (1), (2), (3), and (4) is methoxy or ethoxy.

In embodiment 10, the present invention provides a process according to any of the above embodiments 1, 2, 3, 4, 5, 6, 7, 8, or 9, or any sub-embodiments thereof, wherein R² in each of compounds (1), (2), and (3) is benzyl.

In embodiment 11, the present invention provides a process according to any of the above embodiments 1, 2, 3, 4, 5, 6, 7, 8, or 9, or any sub-embodiments thereof, wherein said compound (1) is obtained via the following order of reactions: (e), (d), (c1), then (a) and (b); or (e), (d), (c2), then (a) and (b).

Alternatively, in a sub-embodiment, said compound (1) is obtained via the following order of reactions: (d), (e), (c1), then (a) and (b); or (d), (e), (c2), then (a) and (b).

Yet alternatively, in another sub-embodiment, said compound (1) is obtained via the following order of reactions: (d), (c1), (e), then (a) and (b); or (d), (c2), (e), then (a) and (b).

In embodiment 11, the present invention provides a compound, which is:

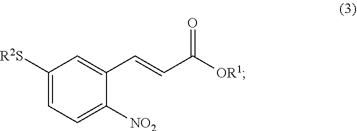

wherein R¹ is ethyl; and R² is benzyl; or a salt thereof.

### DETAILED DESCRIPTION

Unless otherwise stated, the following terms used in the specification and claims are defined for the purposes of this Application and have the following meaning:

“Alkenating agent” means an agent that converts a carbonyl group to alkene group. Two examples of such alkenating agent are “Horner-Wadsworth-Emmons (HWE) agent” and “Wittig agent”. For example, the Wittig reaction uses phosphoranes to convert an α,β-unsaturated ketone to a conjugated alkene as follows:

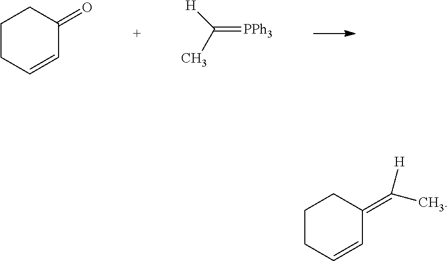

The Wittig reaction produces Z alkenes. Horner-Wadsworth-Emmons (HWE) reaction is a variation of the Wittig reaction, which gives E alkenes. The Z and E alkenes refer to the relative position of the two higher priority groups in an alkene group as widely understood by those skilled in the art. If the higher priority groups are on the same side of the alkene group, then the alkene is called a Z-alkene (German; zusammen=together). If the higher priority groups are on the opposite sides of the alkene group, then the alkene is called an E-alkene (German; entgegen=opposite).

“(C_{α}-C_{β}) Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Amino” means —NH₂.

“(C_{α}-C_{β})Alkoxy” means a —OR radical where R is alkyl as defined above, e.g., methoxy, ethoxy, propoxy, or 2-propoxy, n-, iso-, or tert-butoxy, and the like.

“(C_{α}-C_{β})Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Carboxy” means —COOH.

“GDH-101” means glucose dehydrogenase (GDH) enzyme catalyst that accepts both NAD+ and NADP+ cofactors and is active at temperatures up to 50° C. GDH catalyzes the oxidation of D-glucose to D-glucolactone, while in turn reduces NAD⁺ or NADP⁺ to NADH and NADPH, respectively. The product of this reaction, D-glucolatone, spontaneously and irreversibly hydrolyses in water to gluconic acid, therefore favoring the formation of reduced NADH and NADPH. GDH-101 is commercially available at Matthey.com.

“Halo” or “Halogen” means fluoro, chloro, bromo, or iodo.

The present invention also includes protected derivatives of compounds of Formula (1). For example, when compounds of Formula (1) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons. Inc. (1999), the disclosure of which is incorporated herein by reference in its entirety. The protected derivatives of compounds of Formula (1) can be prepared by methods well known in the art.

A “salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid). 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

“Precious metal catalyst” means a noble metal widely used in the chemical industry owing to their ability to speed up the chemical process. Gold (Au), palladium (Pd), platinum (Pt), rhodium (Rh), ruthenium (Ru), and silver (Au) are some of the examples of precious metals.

### Generic Experimental Procedures

The methods of the present invention can be performed either according to Method A, or Method B below: wherein each of R¹; R²; X; and X₁ are as defined above:

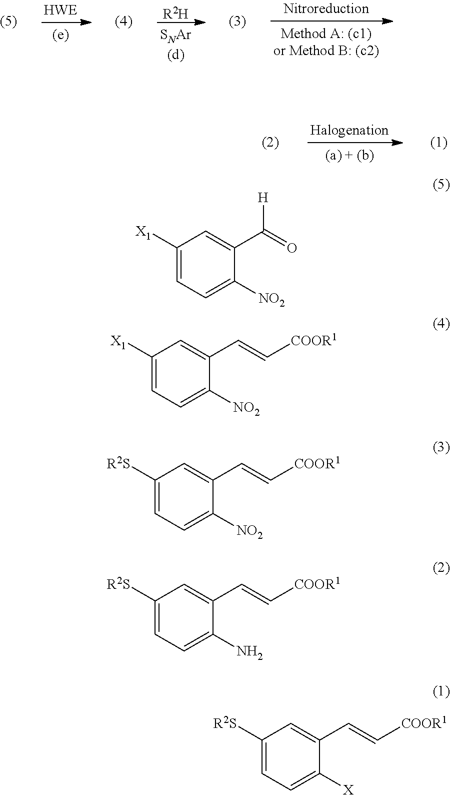

The above methods A and B comprise: Step (e): Horner-Wadsworth-Emmons (HWE); Step (d): aryl substitution (S_{N}Ar); Step (c1): metal mediated or (c2): enzymatic mediated nitroreduction; and Steps (a)+(b): halogenation.

Those skilled in the art would understand that the above Method A and Method B of the present invention can be performed in various orders and are not limited to the orders of the steps described in the generic procedures above. The present inventors contemplate that the order of the reaction steps of the present invention can vary. For example, those skilled in the art understand that the Methods A and B above can be performed as follows Step (d): S_{N}Ar step; Step (e): HWE step; Step (c1) or (c2): metal mediated or enzymatic mediated nitroreduction; and followed by Steps (a)+(b): halogenation to form compound (1).

Alternatively, those skilled in the art understand that the Methods A and B above can be performed as follows Step (d): S_{N}Ar; Step (c1) or (c2): metal mediated or enzymatic mediated nitroreduction; Step (e): HWE; and followed by Steps (a)+(b): halogenation to form compound (1).

Step (e): HWE: The HWE step (e) is a fast step and can be completed in less than 1 h. It is also a clean step that typically produces a high yield compound (4) product.

Step (d): S_{N}Ar: The water content, the charge of Cs₂CO₃, and charge of Bn—SH are very critical parameters in the S_{N}Ar step. The high temperature and long reaction time (10 h to 12 h) are required to drive the observed intermediate compound and the observed bis addition impurity compound to form the compound (3) product. Both the intermediate compound and the bis addition impurity compound can be easily purged by crystallization.

| | | | |
|---|---|---|---|
| (4) | observed intermediate | observed bis-addition | (3) |
| | compound | impurity | |

Step (c1): Fe° is the preferred metal used in the metal mediated nitroreduction step to prepare compound (2). It is hazardous material and the activation is highly exothermic. It is therefore preferred that the iron activation step is conducted before compound (3) is added into the reaction mixture.

Steps (a)+(b): In the halogenation step, thiosulfate quench resulted in generation of elemental sulfur (S8) which contaminated the isolated compound (1) as follows:

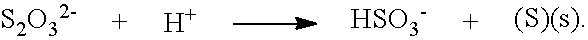

The presence of sulfur was observed to vary in various batches of the halogenation step. The present inventor found that replacing Na-thiosulfate (Na₂S₂O₃) with Na-bisulfite (NaHSO₃) or ascorbic acid eliminated sulfur generation and re-establishes reproducibility and rapid kinetics in C—N bond formation.

The present inventors have found a strategy to suppress key process impurities by controlling reaction temperature (no higher than 5° C.), controlling reaction inertness, and utilizing solvent/AQ mixture defined above (THF, CH₃CN, EtOAc, iPAc). By using these parameters, key process impurities below were minimized and easily purged on isolation. The key impurities and side products are tabulated in Table 1.

TABLE 1
Impurities and Side Products in step (c1);

| | 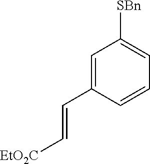 | 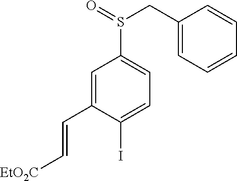 [SMILES: CCOC(=O)/C=C/c1cc(S(=O)Cc2ccccc2)ccc1I] | |
|---|---|---|---|
| | Des-amino | Sulfoxide | Sulfur |
| | Des-Amino Impurity | Sulfoxide Impurity | Elemental Sulfur |
| Cause | H+ reduction of | [O] of sulfide | I₂ quench with thiosulfate |
| | diazonium | | |
| Control Strategy | Temp, 0° C. to 5° C. | Temp, 0° C. to 5° C. | No thiosulfate was used |
| In-Process | 2-6 AP | 0-10 AP | NA |
| Isolated Solids | 0-0.2 AP | 0-0.2 AP | 0-0.78 AP |
| % Purge | 85-92% | >90% | NA |
| Criticality | LOW (yield) | LOW (yield) | HIGH (quality) |
| (yield/quality) | | | |

### Step (c2): The Biocatalytic Nitroreduction Step

The reaction mechanism of the present biocatalytic nitroreduction step is generally depicted as follows:

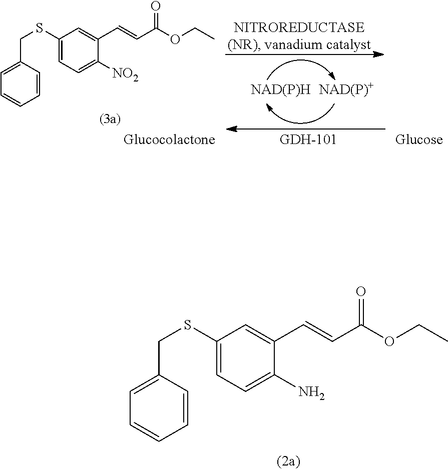 [SMILES: CCOC(=O)/C=C/c1cc(SCc2ccccc2)ccc1[N+](=O)[O-] ; CCOC(=O)/C=C/c1cc(SCc2ccccc2)ccc1N]

### Vanadium Metal Catalyst and Reaction Time

The present inventor found that in the absence of vanadium metal catalyst, more hydroxylamine (HA) side product (m/z=329) having the structure below:

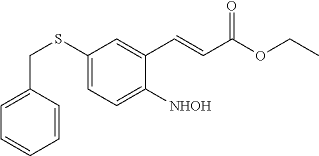

was formed, as evidenced by LCMS and NMR spectra of the reaction mixture. Table 2 listed 19 reaction conditions that were conducted with various enzymatic catalysts to form compound (2a) with and without the metal vanadium catalyst additive at enzyme loading rate of 2 mg/mL.

Reaction protocol for entries #1-10: various nitroreductase (NR) enzymes which were obtained from Johnson Mattey nitroreductase kit, including NR-55, (5 mg) was weighed into 2 ml Eppendorf tubes. A stock solution of buffer was prepared: KPi (250 mM, pH 7, glucose (100 mM), NADP+ (1 mM), GDH (1 mg m L-1) which was added to each enzyme (400 μL) along with 50 μL substrate stock solution (25 mM per reaction) and 50 μL stock V₂O₅ (2 mM per reaction). The reactions were stirred at 35° C., 350 rpm overnight.

Reaction protocol for entries #11-14: NR-55 (10 mg), glucose (90 mg), GDH-101 (5 mg), NADP+ (3.7 mg) were weighed into Radleys carousel tubes equipped with stirrer bar (Fisher PTFE cylindrical, 10×6 mm). 4.0 ml phosphate buffer (pH 7, 250 mM) and 1 ml DMSO (20 vol %) containing 46 mg of compound (3a) and 500 μl stock V₂O₅ (2 mM final concentration) was added to the tubes. Reactions were stirred at 1000 rpm in a Radleys carousel, heated to 45° C. and sampled after 5 h (entries #11-12) and 24 h (entries #13-14).

Reaction protocol for entries #15-18: NR-55 (10 mg), glucose (180 mg), GDH-101 (5 mg), NADP+ (3.7 mg) were weighed into Radleys carousel tubes equipped with stirrer bar (Fisher PTFE cylindrical, 10×6 mm). 4.0 ml phosphate buffer (pH 7, 250 mM) and 1 ml DMSO (20 vol % or 30 vol %) containing 92 mg of compound (3a) and 500 μl stock V₂O₅ (2 mM final concentration) was added to the tubes. Reactions were stirred at 1000 rpm in a Radleys carousel, heated to 45° C. and sampled after 5 h (entries #15-18) and 24 h (entries #15A-18A).

TABLE 2
Various reaction condition testing vanadium metal catalyst in step (c2):

| | Enzyme | Metal | DMSO | [(3a)] | (3a) | (2a) | Other | |
|---|---|---|---|---|---|---|---|---|
| No. | Catalyst | Catalyst | Vol % | mM | (%) | (%) | (%) | HA(%) |
| 1 | NR-4 | V₂O₅ | 10 | 25 | 54 | 36 | 10 | 0 |
| 2 | NR-5 | V₂O₅ | 10 | 25 | 57 | 34 | 9 | 0 |
| 3 | NR-14 | V₂O₅ | 10 | 25 | 54 | 41 | 5 | 0 |
| 4 | NR-17 | V₂O₅ | 10 | 25 | 71 | 28 | 1 | 0 |
| 5 | NR-55 | V₂O₅ | 10 | 25 | 57 | 34 | 9 | 0 |
| 6 | NR-4 | None | 10 | 25 | 30 | 15 | 2 | 53 |
| 7 | NR-5 | None | 10 | 25 | 32 | 14 | 3 | 51 |
| 8 | NR-14 | None | 10 | 25 | 50 | 12 | 0 | 38 |
| 9 | NR-17 | None | 10 | 25 | 44 | 19 | 0 | 37 |
| 10 | NR-55 | None | 10 | 25 | 30 | 12 | 3 | 55 |
| 11 | NR-55 | V₂O₅ | 20 | 25 | 12 | 39 | 13 | 37 |
| 12 | NR-55 | None | 30 | 25 | 5 | 7 | 6 | 82 |
| 13 | NR-55 | V₂O₅ | 40 | 25 | 4 | 59 | 36 | 1 |
| 14 | NR-55 | None | 50 | 25 | 5 | 35 | 60 | 0 |
| 15 | NR-55 | None | 20 | 50 | 11 | 10 | 2 | 77 |
| 16 | NR-55 | None | 30 | 50 | 3 | 8 | 8 | 81 |
| 17 | NR-55 | V₂O₅ | 20 | 50 | 7 | 27 | 18 | 48 |
| 18 | NR-55 | V₂O₅ | 30 | 50 | 3 | 29 | 17 | 51 |
| 15A | NR-55 | V₂O₅ added | 20 | 50 | 9 | 53 | 38 | 0 |
| 16A | NR-55 | V₂O₅ added | 30 | 50 | 5 | 50 | 45 | 0 |
| 17A | NR-55 | V₂O₅ | 20 | 50 | 16 | 41 | 42 | 1 |
| 18A | NR-55 | V₂O₅ | 30 | 50 | 4 | 49 | 46 | 1 |
| 19 | NR-55 | NH₄VO₃ | 30 | 100 | 17 | 66 | 15 | 1 |

HA = Hydroxylamine side product; [(3a)] = compound (3a) concentration.

An improved HPLC method was developed, and subsequent reactions were analyzed using this method. NR-5 was tested with and without V₂O₅ and was analyzed at different time points to check if the method was suitable to detect short-lived reaction intermediates. The reaction was sampled at 1 h, 5 h and 72 h.

After 1 h, the reaction with vanadium did not show any additional peaks, whereas without vanadium, a new peak at 3.98 min (hydroxylamine) appeared. After 5 h, new peaks were observed both with and without vanadium present. A few reaction intermediate compounds were observed. Interestingly, after 72 h, both reactions showed increased conversion to the aniline compound (2a) with a decrease in the peaks at 3.99 min (hydroxylamine). The peak at 3.99 min is the hydroxy lamine and the peak at 5.06 min is the nitroso compounds. LCMS appears to give m/z of 328 and 330, corresponding to the nitroso and hydroxylamine. The later eluting peak at 8.18 min gives m/z=661=[M+Na]+, which corresponds to the mass of azoxy side-product.

The present inventors further tested NR-55 enzyme with and without vanadium catalyst on a 5 mL scale with 2 mg/mL loading rate (see entries #11-14) and found that as observed before, the vanadium influences the initial product distribution. Most notably, after 5h, in the absence of V₂O₅, there was a substantial build-up of the hydroxylamine (82%) and only 7% aniline, whereas with vanadium there was 37% hydroxylamine and 39% aniline formed (see entries #11-12).

After 24 h, the amount of hydroxylamine remaining was negligible both with and without vanadium, which supported that a spontaneous conversion of hydroxy lamine to the aniline and nitro intermediates (aka. Disproportionation or DP) was taking place.

It was found however, that the amount of other side product, which was determined to be azoxy compound, formed in the reaction without vanadium was much higher at 42%. At lower catalyst loadings, the role of vanadium was much more noticeable. It is believed that this is caused by both the rate of disproportionation and nitroso reduction are reduced and the build-up of these intermediates couple together to form the azoxy compound.

The present inventors further tested NR-55 enzyme with and without vanadium catalyst by doubling the substrate concentration to 50 mM (18 g/L) scale while maintaining the 2 mg/mL loading rate (see entries #15-18). Both 20 volume % and 30 volume % of DMSO were tested for comparison at this higher substrate concentration.

After 5 h, without vanadium (see entries Nos. 15 and 16) 77% and 81% hydroxylamine, respectively, was found to be formed. The reactions containing vanadium gave lower amounts of hydroxylamine, but still quite significant amounts 48% to 51%. This was around 10% higher compared to the previous reaction which was run at a lower subrate concentration of 25 mM. With vanadium, there was about 20% more aniline compound (2a) formed after 5 h (see entries 17 and 18). The amount of vanadium was found to be too low for the disproportionation to be effective.

Reaction entries Nos. 17 and 18 were sampled again after 24 h (see entries Nos. 17A and 18A), and 2 mM vanadium was added to reaction entries Nos. 15 and 16 to learn how quickly the disproportionation takes place (see entries Nos. 15A and 16A).

The present inventors unexpectedly found that after 24 h, entries Nos. 15A, 16 A, 17A, and 18A were quite comparable after vanadium were added at 5 h to entries Nos. 15A and 16 A, i.e., 41% to 53% of aniline (2a) was obtained. HPLC reveals that the azoxy compounds were present in the 26% to 35% concentration.

It was found that higher substrate concentrations produced about 10% higher conversion to the azoxy compound after 24 h and there was still nitro starting material compound (3a) remaining after 24 h. The higher build ups of hydroxylamine compound seemed to afford more azoxy compound by the end of the reaction. HPLC showed a dramatic effect of vanadium addition. The bulk of the hydroxylamine compound was converted to the aniline (2a) and the nitroso, which then reacted with the hydroxy lamine to form the azoxy compound. A second HPLC trace was collected 30 mins after the addition of vanadium.

The reaction was intensified by testing 100 mM substrate concentration (37 g/L), which equals to 184 mg in 5 mL. The starting material was added via syringe pump at a rate of 1.5 mL/h (See Entry #19). Larger stirrer bar was used as the smaller ones did not stir very well at this concentration. However, this led to a build-up of sticky gum on the stir bar by the end of the reaction. After 4 h, 63% aniline (3a) had been produced and unexpectedly, no hydroxylamine was formed. After 24 h, 66% aniline (3a) and 7% azoxy were formed, but 17% nitro starting material (2a) remained. While the reaction worked at this higher substrate concentrations, the reaction vessel, type of stirring and mass transfer will need to be considered to avoid the build-up of sticky gum on the stirrer bar.

### pH Effect on the Reaction Condition

The present inventors have tested the present reduction reaction in pH 7 and 8. pH 6 was further tested to learn any effect pH may have on azoxy formation.

Reaction protocol: NR-55 (10 mg), glucose (90 mg), GDH-101 (5 mg), NADP⁺ (3.7 mg) and V₂O₅ (0.1 equiv.) were weighed into Radleys carousel tubes equipped with stirrer bar (Fisher PTFE cylindrical, 10×6 mm). 4.0 mL phosphate buffer (pH 6, 250 mM) and 1 mL (20%) DMSO containing 46 mg of compound (3a) was added to the tubes. Reactions were stirred at 1000 rpm in a Radleys carousel, heated to 45° C. and sampled after 24 h. The various reaction condition at various pH is listed in Table 3:

TABLE 3
Various reaction condition testing optimum pH in step (c2):

| | | | (3a) | (2a) | Azoxy | Other | HA |
|---|---|---|---|---|---|---|---|
| No. | pH | Time | (%) | (%) | (%) | (%) | (%) |
| 1 | 6 | 1 h | 4 | 27 | 14 | 7 | 48 |
| 2 | 6 | 3 h | 10 | 43 | 27 | 8 | 12 |
| 3 | 6 | 24 h | 7 | 45 | 33 | 15 | 0 |

At pH 6, very high levels of azoxy were found to be formed at only 25 mM (33%) after 24 h of reaction time. Therefore pH 7 and 8 are preferred.

### Syringe Pump Addition

Avoiding the build-up of the reaction intermediates seemed key to reducing the amount of azoxy formation. One way to avoid build up is to add the starting material in a fed-batch approach.

The compound (3a) substrate was dissolved in DMSO and was added over 1 h at flow rate of 1 mL/h via a syringe pump. 50 mM substrate concentration was used, 2 mg/mL catalyst loading, 20 vol % DMSO and NH₄VO₃ (0.1 equiv.), since this vanadium salt exhibited better solubility and marginally better conversions. At the end of 1 h, sample was taken after the substrate addition was completed and the present inventors found that there was no build-up of reaction intermediates. After a further hour, 80% conversion to the aniline compound (2a) was achieved and only 8% azoxy was observed on HPLC. The fed-batch approach and the more soluble vanadium source showed a positive effect on the reaction outcome.

A slower flow rate of 0.25 mL/h substrate addition via a syringe pump was tested and after 2 h of reaction time, half of the substrate had been added, and the reaction looked quite clean with mainly compound (3a) (58%) and aniline compound (2) (39%) and a very low amount of reaction intermediates. However, after the remaining substrate was added, and after 24 h, the reaction had not gone to full conversion. There could be stability issues with the NR or the GDH reagents, perhaps one of the enzymes is inactive after this time at 45° C. in 20% DMSO. The higher rate of addition was preferred.

### Metal Catalyst: Amount and Other Metal Testing

Iron and copper were tested in place of the vanadium metal as catalyst. However, vanadium was found to be the preferred catalyst. Vanadium and gold were further tested under the following reaction protocol: NR-55 (10 mg), glucose (90 mg), GDH-101 (5 mg), NADP⁺ (3.7 mg) and V/Au (0.5 equiv.) were weighed into Radleys carousel tubes equipped with stirrer bar (Fisher PTFE cylindrical, 10×6 mm). 4.0 mL phosphate buffer (pH 6, 250 mM) and 1 mL (20%) DMSO containing 46 mg of compound (3a) was added to the tubes. Reactions were stirred at 1000 rpm in a Radleys carousel, heated to 45° C. and sampled after 30 min.

TABLE 4
Various reaction condition testing various metal catalysts in step (c2):

| No. | Metal Catalyst | (3a)(%) | (2a)(%) | Azoxy(%) | Other(%) | HA(%) |
|---|---|---|---|---|---|---|
| 1 | V(IV) oxide phthalocyanine | 4 | 17 | 7 | 3 | 69 |
| 2 | V(IV) oxide bis (2,4-pentanedionate) | 17 | 38 | 15 | 6 | 23 |
| 3 | Vanadyl sulfate hydrate | 14 | 18 | 4 | 1 | 63 |
| 4 | V (V) oxy triethoxide | 17 | 27 | 7 | 3 | 46 |
| 5 | 3% V(Canceled) | 9 | 25 | 13 | 7 | 46 |
| 6 | Gold (III) chloride | 100 | 0 | 0 | 0 | 0 |
| 7 | V (III) 2,4-pentanedionate | 7 | 61 | 17 | 9 | 6 |

After 30 min, V (III) 2,4-pentanedionate showed good conversion to the aniline (2a) (61%) and only 6% hydroxylamine compound (see Table 4, Entry 7). Gold (III) chloride was found to completely inhibited the reaction under the above protocol, showing not even a trace of any of the reaction intermediates (Entry 6).

The reactions were continued to run and samples were taken again after 5 h and 24 h. The results are shown in Tables 5 and 6 below:

TABLE 5
Results for vanadium (and gold) screening after 5 h.

| No. | Metal Catalyst | (3a)(%) | (2a)(%) | Azoxy(%) | Other(%) | HA(%) |
|---|---|---|---|---|---|---|
| 1 | V(IV) oxide phthalocyanine | 4 | 27 | 15 | 6 | 48 |
| 2 | V(IV) oxide bis (2,4-pentanedionate) | 5 | 53 | 21 | 19 | 0 |
| 3 | Vanadyl sulfate hydrate | 4 | 42 | 11 | 7 | 36 |
| 4 | V (V) oxy triethoxide | 6 | 64 | 18 | 9 | 3 |
| 5 | 3% V(Canceled) | 6 | 43 | 27 | 19 | 5 |
| 6 | Gold (III) chloride | 100 | 0 | 0 | 0 | 0 |
| 7 | V (III) 2,4-pentanedionate | 5 | 64 | 18 | 13 | 1 |

After 5 h, V (III) 2,4-pentanedionate gave 66% conversion to aniline compound (2a) (Entry 7). After 5 h, the result with vanadium oxy triethoxide appeared comparable with vanadium (III) acetylacetonate. There was a significant (19%) build-up of nitroso intermediate with vanadium (IV) acetylacetonate (Entry 2) and with vanadium on carbon (Entry 5).

TABLE 6
Results for vanadium (and gold) screening after 24 h.

| No. | Metal Catalyst | (3a)(%) | (2a)(%) | Azoxy(%) | Other(%) | HA(%) |
|---|---|---|---|---|---|---|
| 1 | V(IV) oxide phthalocyanine | 5 | 40 | 7 | 18 | 2 |
| 2 | V(IV) oxide bis (2,4-pentanedionate) | 6 | 55 | 15 | 18 | 0 |
| 3 | Vanadyl sulfate hydrate | 4 | 59 | 4 | 15 | 1 |
| 4 | V (V) oxy triethoxide | 5 | 68 | 7 | 9 | 2 |
| 5 | 3% V(Canceled) | 7 | 43 | 13 | 16 | 1 |
| 6 | Gold (III) chloride | 99 | 0 | 0 | 1 | 0 |
| 7 | V(III) 2,4-pentanedionate | 2 | 66 | 17 | 13 | 1 |

After 24 h, vanadium (III) pentanedionate and vanadium (V) oxy triethoxide showed the highest conversion to the aniline compound (2a).

The present inventors increased the amount of vanadium metal catalyst from 0.1 eq. to 1.0 eq. to learn if the amount of metal catalyst affects the rate of disproportionation. Further an alternative source of vanadium, NH₃VO₄, was tested. NH₃VO₄ was found to have higher solubility compared to V₂O₅.

Reaction protocol: NR-55 (10 mg), glucose (90 mg), GDH-101 (5 mg), NADP+ (3.7 mg) and V₂O₅ (0.1 to 1.0 equiv.) or NH₃VO₄ (0.1 to 1.0 equiv.) were weighed into Radleys carousel tubes equipped with stirrer bar (Fisher PTFE cylindrical, 10×6 mm). 4.0 ml phosphate buffer (pH 7, 250 mM) and 1 ml DMSO (20 vol %) containing 46 mg of compound (3a) was added to the tubes. Reactions were stirred at 1000 rpm in a Radleys carousel, heated to 45° C. and sampled after 24 h.

TABLE 7
Various reaction condition testing the optimum amount
of V₂0₅ and NH₃VO₄ catalyst in step (c2):

| | | Metal | | | | | |
|---|---|---|---|---|---|---|---|
| | | Catalyst | | | | | |
| | Metal | Amount | (3a) | (2a) | Azoxy | Other | HA |
| No. | Catalyst | (eq.) | (%) | (%) | (%) | (%) | (%) |
| 1 | V₂0₅ | 0.1 | 2 | 63 | 22 | 10 | 3 |
| 2 | V₂0₅ | 0.2 | 2 | 66 | 21 | 9 | 2 |
| 3 | V₂0₅ | 0.5 | 2 | 68 | 20 | 9 | 1 |
| 4 | V₂0₅ | 1 | 3 | 73 | 20 | 4 | 0 |
| 5 | NH₃VO₄ | 0.1 | 2 | 63 | 21 | 11 | 3 |
| 6 | NH₃VO₄ | 0.2 | 6 | 66 | 16 | 9 | 3 |
| 7 | NH₃VO₄ | 0.5 | 3 | 71 | 15 | 9 | 2 |
| 8 | NH₃VO₄ | 1 | 2 | 75 | 15 | 5 | 3 |

### Enzymatic Catalyst: Nitro Reductase (NR) and Co-Solvent

The present inventor found that in the previous experiments, 10 vol % DMSO was used as co-solvent, however when the stock solution was added to the aqueous reaction conditions, the solution turned cloudy, suggesting that the starting material was not very soluble in DMSO. In these experiments, the enzymes that gave >40% conversion by HPLC with 10% DMSO, were tested in 10% toluene as co-solvent. In the initial stages of the reaction, there was a clear biphasic system and the aqueous layer was transparent. After an hour, the aqueous layer became cloudier, and after being shaken overnight, the solution was completely cloudy.

Reaction protocol: NR (5 mg) was weighed into 2 ml Eppendorf tubes. A stock solution of buffer was prepared: KPi (250 mM, pH 7, glucose (100 mM), NADP+ (1 mM), GDH (1 mg ml⁻¹) which was added to each enzyme (400 μl) along with 50 μl substrate stock solution in toluene (25 mM per reaction) and 50 μl stock V₂O₅ (2 mM per reaction). The reactions were stirred at 35° C. for 24 h. Reactions were diluted with 1 ml MeCN, vortexed and centrifuged and a 1 ml aliquot was removed and analyzed by HPLC. The conversions are based on uncorrected LCAP at 254 nm. 17 reaction conditions were conducted with various nitroreductase enzymatic catalysts to form compound (2a). The solubility and the mass transfer of the starting material appear to be limiting the reaction.

Under the above reaction conditions, the present inventors found that other niroreductase enzymes gave at least or higher than 30% conversion to aniline compound (2a).

### Co-Solvent Screening at Higher Reaction Temperature of 45° C.

One way to increase solubility is to run the reaction at a higher temperature. NR-55 is a kit enzyme originating from a thermophilic organism. Typically, these enzymes can tolerate higher temperatures and are more resilient to higher volumes of co-solvent. NR-55 was tested in DMSO and toluene (10-30 vol %), pH 7 and at a higher temperature of 45° C. The reactions were carried out on a 5 ml scale using a Radley's Carousel using high stirring speed of 1000 rpm to aid mixing.

Reaction protocol: NR-55 (50 mg), glucose (90 mg), GDH-101 (5 mg), NADP+ (3.7 mg) were weighed into 6 Radleys carousel tubes equipped with stirrer bar (Fisher PTFE cylindrical, 10×6 mm). For 10 vol % co-solvent, 4.5 ml phosphate buffer (pH 7, 250 mM) was added to the tube and 0.5 ml DMSO containing 46 mg of compound (3a) and 500 μl V₂O₅ (2 mM final concentration). The reaction mixture was stirred at 1000 rpm in a Radleys carousel, and heated to 45° C. for 24 h. The mixture was then diluted with 5 ml acetonitrile, stirred and centrifuged and analyzed by HPLC. For 20 and 30 vol %, the volumes were adjusted accordingly.

Table 8 lists 6 reaction conditions that were conducted to test toluene and DMSO as co-solvent at 45° C. to form compound (2a):

TABLE 8
Toluene and DMSO co-solvent testing results
at 45° C. reaction temperature.

| | | Volume | (3a) | (2a) | Azoxy | Other |
|---|---|---|---|---|---|---|
| No. | Co-Solvent | % | (%) | (%) | (%) | (%) |
| 1 | Toluene | 10 | 96 | 4 | 0 | 0 |
| 2 | Toluene | 20 | 93 | 6 | 1 | 0 |
| 3 | Toluene | 30 | 92 | 7 | 0 | 0 |
| 4 | DMSO | 10 | 17 | 68 | 6 | 9 |
| 5 | DMSO | 20 | 0 | 82 | 5 | 13 |
| 6 | DMSO | 30 | 0 | 84 | 6 | 10 |

NR-55 was found to give very low conversions to the desired aniline (2a) with toluene as the co-solvent (Table 8, Entries 1-3). With 20 to 30 vol % DMSO however, the nitro compound (3a) was completely consumed and high levels of aniline (2a) formation was observed (82-84% by LCAP at 254 nm, Table 8, Entries 5 and 6). When only 10 vol % DMSO was used, there was still 17% of starting material remaining, suggesting that the substrate solubility plays an important role in the reaction. These conversions are also much higher than those seen thus far, suggesting that a combination of higher volumes of co-solvent, more efficient stirring and a higher temperature may be beneficial to the reaction. Reaction Entry No. 5 was further extracted with EtOAc (10 ml×2), dried over anhydrous MgSO₄, filtered, and concentrated in vacuo and re-analyzed by HPLC and NMR to ensure the reaction sampling was representative of the reaction mixture.

Given the success of using 20% to 30% volume of DMSO, the present inventors tested NR-55 and NR-5 in parallel at reduced catalyst loadings (1000 rpm) and at pH 7 and 8 and keeping the reaction temperature of 45° C. The reactions were sampled after 5 hours, then again after 24 h.

Reaction protocol: NR-5 or NR-55 (10-50 mg), glucose (90 mg), GDH-101 (5 mg), and NADP+ (3.7 mg) were weighed into 12 Radleys carousel tubes equipped with stirrer bar (Fisher PTFE cylindrical. 10×6 mm). 4 ml phosphate buffer (pH 7 or 8, 250 mM) was added to the tube and 1 ml DMSO containing 46 mg of compound (3a) and 500 μl V₂O₅ (2 mM final concentration). The reactions were stirred at 1000 rpm in a Radleys carousel, heated to 45° C. for 5 h and 24 h. Reactions samples A 100 μl aliquot reaction sample was taken, 500 μl MeCN was added, vortexed, centrifuged and analyzed by HPLC. Some nitro compound (3a) was found to remain, but it was a shoulder peak.

After 5 h, NR-55 appeared to consume starting compound (3a) compared to NR-5, preferably at 2 mg/mL substrate loading rate.

A lower temperature of 35° C. was further tested under the following reaction protocol: NR-55 (10 mg), glucose (90 mg), GDH-101 (5 mg), NADP⁺ (3.7 mg) and NH₄VO₃ (1 equiv.) were weighed into Radleys carousel tube equipped with stirrer bar (Fisher PTFE cylindrical, 10×6 mm). 3.5 mL phosphate buffer (pH 7, 250 mM) and 1.5 mL (20%) DMSO containing 46 mg of compound (3a) was added to the tubes via syringe pump addition at 1.5 mL/h rate. Reactions were stirred at 1000 rpm in a Radleys carousel, heated to 45° C. and sampled after 1, 2, 4 and 24 h.

It was found that the reaction worked at 35° C. reaction temperature. 6% compound (3a) was found to remain and 15% azoxy was found at the end of the 24 h reaction.

### Buffer Testing

The reaction had previously only been performed in phosphate buffer, which is one of the most common buffers and is economical to scale up. A buffer screen was carried out to see if any alternatives would give a better reaction profile. 10 buffers were screened in parallel at 100 mM concentration, using substrate concentration of 25 mM and various pH conditions.

Reaction protocol: NR-55 (10 mg), glucose (96 mg), GDH-101 (5 mg), NADP⁺ (3.7 mg) and NH₄VO₃ (15 mg, 1 equiv.) were weighed into Radleys carousel tubes equipped with stirrer bar. 3.5 mL buffer (pH 5-10, 100 mM) and 1.5 mL (30%) DMSO containing 46 mg compound (3a). The reactions were heated to 45° C. and sampled after 2 h and 24 h.

The reaction samples were taken after 2 h and 24 h. The results are shown in Tables 9 and 10 below:

TABLE 9
Results for buffer screening in step (c2) after 2 h.

| No. | Buffer | (3a)(%) | (2a)(%) | HA(%) | Nitroso | Azoxy(%) | Other(%) |
|---|---|---|---|---|---|---|---|
| 1 | Acetate pH 5 | 95 | 4 | 0 | 0 | 1 | 0 |
| 2 | PIPES pH 6 | 55 | 35 | 0 | 0 | 8 | 2 |
| 3 | Bicine pH 8 | 9 | 72 | 1 | 0 | 13 | 6 |
| 4 | TES pH 8 | 7 | 69 | 0 | 4 | 14 | 6 |
| 5 | HEPES pH 8 | 7 | 69 | 0 | 4 | 14 | 6 |
| 6 | Tricine pH 8 | 10 | 78 | 0 | 0 | 9 | 3 |
| 7 | Tris pH 8 | 16 | 68 | 0 | 3 | 8 | 5 |
| 8 | CHES pH 8 | 5 | 73 | 0 | 4 | 10 | 8 |
| 9 | CAPS pH 10 | 15 | 67 | 0 | 4 | 7 | 7 |
| 10 | KPi pH 7 | 10 | 53 | 1 | 6 | 16 | 14 |

TABLE 10
Results for buffer screening in step (c2) after 24 h.

| No. | Buffer | (3a)(%) | (2a)(%) | HA(%) | Nitroso | Azoxy(%) | Other(%) |
|---|---|---|---|---|---|---|---|
| 1 | Acetate pH 5 | 97 | 1 | 0 | 0 | 1 | 1 |
| 2 | PIPES pH 6 | 44 | 31 | 0 | 0 | 8 | 17 |
| 3 | Bicine pH 8 | 6 | 74 | 2 | 0 | 10 | 8 |
| 4 | TES pH 8 | 2 | 69 | 1 | 1 | 15 | 12 |
| 5 | HEPES pH 8 | 3 | 77 | 1 | 1 | 10 | 8 |
| 6 | Tricine pH 8 | 4 | 89 | 0 | 0 | 8 | 0 |
| 7 | Tris pH 8 | 2 | 74 | 2 | 0 | 12 | 10 |
| 8 | CHES pH 8 | 3 | 77 | 2 | 0 | 11 | 7 |
| 9 | CAPS pH 10 | 16 | 67 | 2 | 0 | 7 | 8 |
| 10 | KPi pH 7 | 10 | 59 | 2 | 0 | 19 | 10 |

The present inventors found that after 24 h, TRICINE buffer produced 89% compound (2a) product, 8% azoxy and only 4% starting material (3a) (Table 10, Entry 6). Acetate and PIPES buffers produced the lowest conversion to compound (2a) (Entries 1 and 2). BICINE, HEPES, TRIS and CHES also performed well at higher than 70% conversion to compound (2a) after 24 h.

The following are chemical structures of the buffers that were used in the reactions of the present invention:

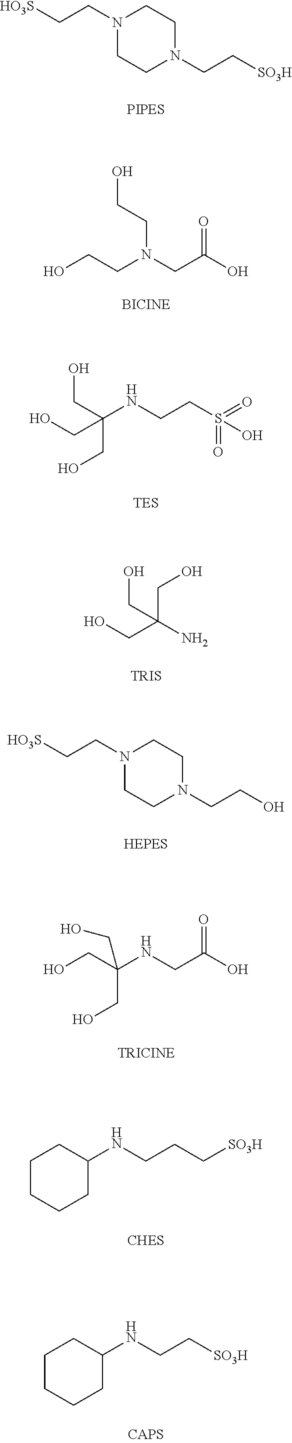

The present inventors ran the reactions in TRICINE buffer (100 mM) with fed-batch addition of the starting material compound (3a). No pH control was used in this reaction to see how much the pH decreased with the reaction progression, and the effect which it might have.

Reaction protocol: NR-55 (10 mg), glucose (386 mg), GDH-101 (5 mg), NADP⁺ (3.7 mg) and NH₄VO₃ (1 equiv.) were weighed into Radleys carousel tube equipped with stirrer bar. 3.5 mL TRICINE buffer (pH 8, 100 mM) and 1.5 mL (30%) DMSO containing 184 mg compound (3a) was added to the tube via syringe pump addition at 1.5 mL/h rate. The reaction was stirred at 500 rpm, heated to 45° C. and sampled after 2, 4 and 24 h.

TABLE 11
Various reaction condition testing the optimum amount
of V₂0₅ and NH₃VO₄ catalyst in step (c2):

| No. | Buffer | (3a)(%) | (2a)(%) | HA(%) | Nitroso | Azoxy(%) | Other(%) |
|---|---|---|---|---|---|---|---|
| 1 | Tricine-2 h | 40 | 59 | 0 | 0 | 1 | 0 |
| 2 | Tricine-4 h | 5 | 91 | 0 | 3 | 2 | 0 |
| 3 | Tricine-24 h | 11 | 85 | 0 | 0 | 2 | 2 |

It was found that 100 mM of TRICINE buffer resulted in low levels of azoxy formation (2%). The pH was measured at the end of the reaction and had dropped to pH 5.7. The reaction mixture was not homogenous, there appeared to be oily gum precipitating out of solution onto the stirrer bar and some yellow solid on the walls of the tube. After the reaction, the reaction mixture was transferred into a vial and centrifuged and both the aqueous and the pellet were analysed to understand the composition. Additionally, the stirrer bar gum and the yellow precipitate was dissolved in acetonitrile and analysed by HPLC. It was found that the solid in the suspension was 90% aniline compound (2a). Both the stirrer bar and the wall of the glass contained more of the nitro starting material (3a) and the gum had also trapped some of the nitroso intermediate.

The reaction was repeated in TRICINE buffer of 250 mM strength and NaOH (10 M) was added manually when the pH dropped during the reaction, maintaining the pH at 8 throughout the reaction.

Reaction protocol: NR-55 (10 mg), glucose (386 mg), GDH-101 (5 mg), NADP⁺ (3.7 mg) and NH₄VO₃ (1 equiv.) were weighed into Radleys carousel tube equipped with stirrer bar. 3.5 mL TRICINE buffer (pH 8, 250 mM) and 1.5 mL (30%) DMSO containing 184 mg compound (3a) was added to the tube via syringe pump addition at 1.5 mL/h rate. pH was kept at 8 using 10 M NaOH (˜70 μL). The reaction was stirred at 500 rpm, heated to 45° C. and sampled after 1, 3, 5 and 24 h.

After 24 h, by HPLC analysis the reaction composition was 84% aniline compound 2a, 3% azoxy, and 4% nitro. There was precipitate found on the walls and an oily gum caked the stirrer bar.

TABLE 12
Various reaction condition testing the buffer and reaction timing in step (c2):

| No. | Buffer | (3a)(%) | (2a)(%) | HA(%) | Nitroso | Azoxy(%) | Other(%) |
|---|---|---|---|---|---|---|---|
| 1 | Tricine-1 h | 30 | 69 | 0 | 0 | 1 | 0 |
| 2 | Tricine-3 h | 35 | 62 | 0 | 0 | 1 | 2 |
| 3 | Tricine-5 h | 6 | 85 | 0 | 6 | 2 | 1 |
| 4 | Tricine-24 h | 4 | 84 | 0 | 3 | 3 | 6 |

Based on the above reaction conditions, the present inventors have shown that the nitro reduction catalytic reactions can be conducted under many different conditions to convert nitro aromatic compound (3a) to aniline compound (2a). Variables of the reaction conditions that can be used include, but not limited to, (1) nitroreductase enzyme, preferably NR-55: (2) reaction temperature, preferably 45° C.; (3) solvent and solvent concentration, preferably 30 vol % DMSO; (5) 2 mg/mL catalyst loading rate; and (6) a metal catalyst, preferably vanadium metal, such as V₂O₅ or NH₃VO₄. A fed-bath approach to substrate addition, coupled with an increased concentration of a soluble vanadium metal source, minimised the build-up of the hydroxy lamine and nitroso intermediates, which led to a decrease in azoxy formation. These side-product compounds were further reduced by using (7) a buffer, preferably TRICINE buffer. The reaction was demonstrated on a 1 g scale at 40 g/L substrate concentration with 2 g/L catalyst loading rate.

The present inventors understand that the nitroreduction reaction can be intensified further by using the suitable the reactor type, including the stirring mechanism, since the limiting factor appears to be the low solubility of the starting material, which leads to the formation of a sticky gum on the stirrer bar. Use of various reactor types to avoid the build-up of sticky gum on the stirrer bar is therefore contemplated to be within the scope of the present invention. Furthermore, use of a second co-solvent, surfactants or deep eutectic solvents/ionic liquids to aid solubility of the starting material is therefore contemplated to be within the scope of the present invention.

The present inventors further understand that the rate of disproportionation of the hydroxy lamine compound is key to minimising the azoxy side-product formation in the absence of the enzyme. Use of various ways to optimize the hydroxy lamine compound rate of disproportionation is therefore contemplated to be within the scope of the present invention.

The present inventors further understand that the disproportionation in TRICINE buffer appears fast. Therefore, use of reduced amount of vanadium metal catalyst to lower than 1 equivalent is contemplated to be within the scope of the present invention.

The present inventors further understand that a one-pot reaction may be of interest in the long run. Therefore, immobilisation of the nitroreductase enzyme is contemplated to be within the scope of the present invention.

The invention will now be described in reference to the following specific Examples. These examples are not to be regarded as limiting the scope of the present invention, but shall only serve in an illustrative manner.

The following abbreviations are used throughout the description and appended claims, and they have the following meanings:

“AP” means Area Percent, which refers to an area under the peak as measured by liquid or gas chromatograms. The AP is a function of a compound concentration in the sample. Below is an example of GC report, wherein % Area represents the AP of each of the named compounds:

| | Name | Retention Time | Area | % Area | Height |
|---|---|---|---|---|---|
| 1 | aniline | 1.919 | 1094751 | 89.13 | 1431757 |
| 2 | Hydroxyl- | 1.954 | 6693 | 0.54 | 7281 |
| | amine | | | | |
| 3 | unknown | 2.005 | 15391 | 1.25 | 21542 |
| 4 | nitro | 2.046 | 83190 | 6.77 | 115442 |
| 5 | azoxy | 2.434 | 28231 | 2.30 | 33356 |

“Ar” means aryl.

“cmp” means compound or compounds.

“CH₃CN” or “MeCN” means acetonitrile.

“CPME” means cyclopropyl methyl ether.

“DMAc” or “DMA” means dimethylacetamide.

“DMF” means dimethylformamide.

“DCM” means dichloromethane.

“DMSO” means dimethylsulfoxide

“EtOAc” means ethyl acetate.

“h” means hour or hours

“HPLC” means high performance liquid chromatography

“IPA” means isopropyl alcohol.

“IPAc” means isopropyl acetate.

“IPC” means in-process control, which is routine checks that are performed during process development. The function of in-process control is monitoring and if necessary, adaption of the manufacturing processes to ensure that the product conforms to its specification. For example, if the target product conversion is 98%, if IPC fails, then a recourse such as longer hold reaction time or additional reagent charge is performed.

“KF” means Karl Fischer titration value, which is a titration method that uses volumetric or coulometric titration to determine the quantity of water present in cach analyte as measured by a Karl Fischer titrator. The chemicals used in the synthetic routes delineated herein include, for example, solvents, reagents, and catalysts. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989): T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

“LCMS” means liquid chromatography mass spectrometry

“LiCI” means lithium chloride

“MIBK” means methyl isobutyl ketone

“mins” means minutes

“MSA” means methane sulfonic acid (MeSO₃H).

“MTBE” means methyl tertiary-butyl ether.

“NMP” means N-methyl-2-pyrrolidone

“Ph” means phenyl.

“ppm” means parts per million, unit of a concentration.

“rt” or “RT” means room temperature

“temp” means temperature

“THF” means tetrahydrofuran

### Examples: Experimental Procedures

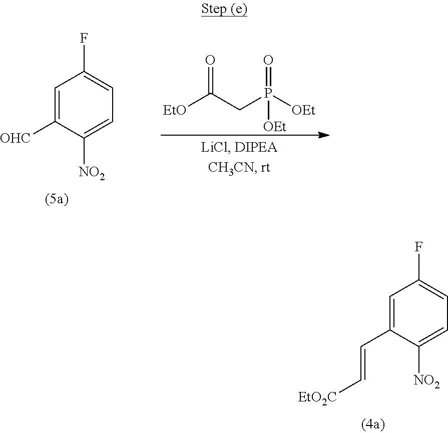 [SMILES: O=Cc1cc(F)ccc1[N+](=O)[O-] ; CCOC(=O)CP(=O)(OCC)OCC ; CCOC(=O)/C=C/c1cc(F)ccc1[N+](=O)[O-]]

### Example 1

Step (e): Preparation of ethyl (E)-3-(5-fluoro-2-nitrophenyl) acrylate (Compound 4a)

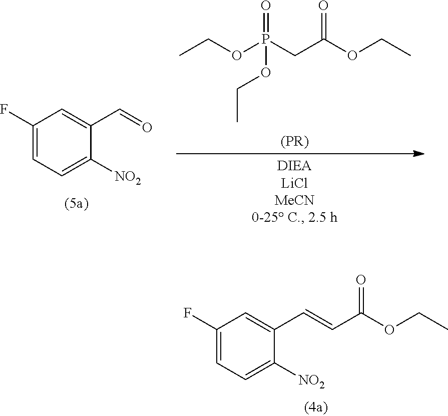 [SMILES: O=Cc1cc(F)ccc1[N+](=O)[O-] ; CCOC(=O)CP(=O)(OCC)OCC ; CCOC(=O)/C=C/c1cc(F)ccc1[N+](=O)[O-]]

Reaction procedure: An inert vessel A was purged under nitrogen gas three times. The reactor jacket temperature was set to 25±5° C. Acetonitrile (7.0 V, 343 kg) was added to vessel A, followed by N,N-diisopropylethylamine/Hünig's base (DIEA) (1.1 eq, 52.1 kg), and the mixture was agitated for 10 minutes. LiCl (2.0 eq, 31.1 kg) was then added and the mixture was agitated again for 10 minutes. The temperature was then cooled down to reach 0° C. internal temperature. The Phosphoryl Reagent (PR) ethyl 2-(diethoxyphosphoryl) acetate (1.05 eq, 86.2 kg) was then added to the mixture, while maintaining internal temperature between 0° C. to 5° C. Slow rise in temperature was observed and the reaction fluid was white and cloudy. The reaction mixture was then heated to reach 25° C. internal temperature and was stirred at 25° C. for 30 minutes. The reaction mixture was then cooled down to reach 0° C. internal temperature. Starting material compound 5-fluoro-2-nitrobenzaldehyde (Compound 5a, 1.0 eq, 62.0 kg) was then slowly added while maintaining internal temperature between 0° C. to 5° C. Slow rise in temperature was observed and the reaction fluid changed from white turbid to brown clear color. The reaction mixture was then heated to reach 25° C. internal temperature and the mixture was stirred at 25° C. for 2 hours. The reaction fluid was observed to change from brown to white and it thickened. HPLC showed the starting material (5a) was consumed and about 97.0% (220 nm) of the product ethyl (E)-3-(5-fluoro-2-nitrophenyl)acrylate (Compound 4a, 3.216 min) was formed. Water (1.0 V, 62.0 L) was then added to the mixture and the reaction fluid changed from thick white to brown clear. The mixture was agitated for 10 minutes.

TABLE 13
Optimization condition of step (e) with 1 kg,
10 kg, and 62 kg scale starting material (5a):

| | | | Condition | | | | | |
|---|---|---|---|---|---|---|---|---|
| | Compound | | PR | LiCl/DIEA | | Time | | |
| Entry | (5a) (g) | | (eq.) | (eq.) | T(° C.) | (h) | Solvent | Weight (g) |
| 1 | 1.00 | kg | 1.20 | LiCl (2.0 eq), | 0° C. | 2.5 | MeCN | 1.10 kg product |
| | | | | DIEA (1.1 eq) | to | | (7.00 V) | and 550 g crude |
| | | | | | 25° C. | | | from mother |
| | | | | | | | | liquor |
| 2 | 10.0 | kg | 1.05 | LiCl (2.0 eq), | 0° C. | 2.5 | MeCN | 87.6 kg, |
| | | | | DIEA (1.1 eq) | to | | (7.00 V) | 99.3% purity, |
| | | | | | 25° C. | | | 78.3% yield; |
| | | | | | | | | QNMR: 91.8% |
| 3 | 62.0 | kg | 1.05 | LiCl (2.0 eq), | 0° C. | 2.5 | MeCN | KF = 0.3-0.4% |
| | | | | DIEA (1.1 eq) | to | | (7.00 V) | (5 points) |
| | | | | | 25° C. | | | |

PR = Phosphoryl Reagent, which is ethyl 2-(diethoxyphosphoryl)acetate

Work-up procedure: 10 kg scale and 62 kg scale were combined for workup. Methyl tertiary butyl ether (MTBE) (3.0 V, 160 kg) was added to the mixture. The two phases were separated, and the aqueous layer was extracted once with MTBE (2.0 V, 120 kg). The organic phases were then combined and washed with saturated NH₄OAc (2.0 V, 160 kg). The organic layer was then concentrated to about 3.0 V.

Distillation procedure: Isopropyl acetate (iPAC) (192 kg) was added to the mixture and concentrated to about 3.0 V by atmospheric distillation. This was done twice to ensure solvent swap complete and water removal.

Isolation procedure: n-Heptane (10 V, 492 kg) was added and the mixture was cooled down to reach −10° C. internal temperature. The mixture was then stirred at −10° C. for 6 hours. The solid was filtered with and the filter cake was washed with n-heptane (2.0 V, 98 kg) and cooled to −10° C. The solid was then dried under N₂ gas. White solid; (87.6 kg, 99.3% purity, 78.3% yield; QNMR: 91.8%). Mp: 59° C. ¹H NMR (500 MHz, chloroform-d) δ 8.15-8.10 (m, 2H), 7.32-7.21 (m, 2H), 6.35 (d, J=15.8 Hz, 1H), 4.30 (q, J=7.1 Hz, 2H), 1.36 (t, J=7.1 Hz, 3H).

### Example 2

Step (d): Preparation of ethyl(E)-3-(5-(benzylthio)-2-nitrophenyl)acrylate (Compound 3a)

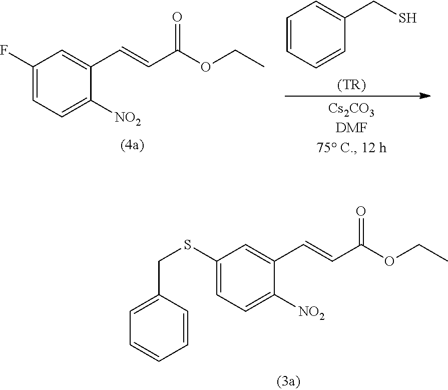

Reaction procedure: An inert vessel A was purged under nitrogen gas three times. The reactor jacket temperature was set to 25±5° C. DMF (5.0 V, 337.5 L) was charged to vessel A, and the vessel was again purged under nitrogen gas three times. Starting material compound ethyl (E)-3-(5-fluoro-2-nitrophenyl)acrylate (Compound 2a, 1.0 eq, 67.5 kg) was then added and the mixture was agitated for 20 minutes. Cs₂CO₃ (1.0 eq, 86.5 kg) was slowly added and the reaction fluid was observed to change from yellow to black. The mixture was agitated for 20 minutes, and the vessel was again purged under nitrogen gas three times. Thiol reagent (TR) compound phenyl methanethiol (1.0 eq, 33.6 kg) was slowly added to the mixture and the temperature rose slowly due to exothermic reaction. The mixture was agitated for 10 minutes and checked by KF (KF: 0.12%). The mixture was then heated to reach 75° C. internal temperature and was stirred at 75° C. for 12 hours. HPLC showed the starting material Compound (4a) was consumed (3.216 minutes) and about 92.6% of the product (E)-3-(5-(benzylthio)-2-nitrophenyl)acrylate (Compound (3a), 4.139 minutes) was formed. Water (5.0 V, 337.5 L) was then added and the reaction fluid was observed to change from thick white to brown clear. The mixture was then agitated for 10 minutes.

TABLE 14
Optimization condition in step (d) with 1.1 kg,
20 kg, and 67.5 kg scale starting material (4a):

| | | | Condition | | | | | |
|---|---|---|---|---|---|---|---|---|
| | Cmp | | TR | LiCl/DIEA | | Time | | |
| Entry | (4a) (g) | | (eq.) | (eq.) | T(° C.) | (h) | Solvent | Weight (g) |
| 1 | 1.10 | kg | 1.00 | Cs₂CO₃ | 70-80 | 12 + 4 | DMF | 1.32 kg product |
| | | | | (1.0 eq) | | | (5.00 V) | (82.75% yield) and |
| | | | | | | | | 180 g crude from |
| | | | | | | | | mother liquor |
| 2 | 20.0 | kg | 1.00 | Cs₂CO₃ | 70-80 | 12 | DMF | 102 kg |
| | | | | (1.0 eq) | | | (5.00 V) | (97.2% purity, |
| 3 | 67.5 | kg | 1.00 | Cs₂CO₃ | 70-80 | 12 | DMF | 86.0% yield) |
| | | | | (1.0 eq) | | | (5.00 V) | |

PR = thiol reagent, which is phenyl methanethiol

Work-up procedure: 20 kg scale and 67.5 kg scale were combined for workup. Methyl tertiary butyl ether (MTBE) (5.0 V, 675 L) was added to the mixture. The two phases were separated and the aqueous layer was extracted once with MTBE (3.0 V, 405 L). The organic phases were then combined and washed with water (5.0 V, 675 L). The organic layer was then concentrated to about 3.0 V.

Distillation procedure: Isopropyl acetate (IPAC) (192 kg) was added to the mixture and concentrated to about 3.0 V by heating.

Isolation procedure: n-Heptane (9.0 V, 1215 L) was added and the mixture was stirred at 25° C. for 6 hours. The solid was filtered with and the filter cake was washed with n-heptane (2.0 V, 270 L). The solid was then dried under N₂ gas. Yellow solid: (102 kg, 97.2% purity, 86.0% yield). Mp: 73° C. ¹H NMR (500 MHZ, chloroform-d) δ 8.11 (d, J=15.8 Hz, 1H), 7.97 (d, J =8.6 Hz, 1H), 7.39-7.27 (m, 7H), 6.20 (d, J=15.8 Hz, 1H), 4.28 (q, J=7.1 Hz, 2H), 4.25 (s, 2 H), 1.35 (t, J=7.1 Hz, 3H).

### Example 3a

Step (c1): Preparation of ethyl (E)-3-(5-(benzylthio)-2-nitrophenyl)acrylate (Compound 2a) via Iron and NH₄Cl Reduction

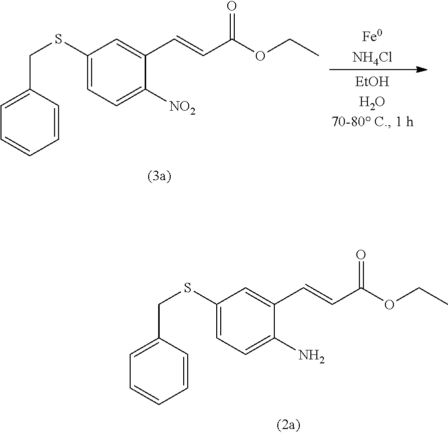 [SMILES: CCOC(=O)/C=C/c1cc(SCc2ccccc2)ccc1[N+](=O)[O-] ; CCOC(=O)/C=C/c1cc(SCc2ccccc2)ccc1N]

Three reactions were carried out in parallel. (34.0 kg×3)

Reaction procedure: A reactor jacket containing an inert vessel temperature was set to 25±5° C. EtOH (4.0 V, 136 L) was charged to the vessel. Water (2.0 V, 68.0 L) was then added to the vessel, followed by NH₄Cl (5.0 eq, 26.5 kg). The mixture was then agitated for 10 minutes. Fe° (3.0 eq, 16.6 kg) was then slowly added and the mixture was agitated for 10 minutes. The mixture was then heated to reach 70° C. internal temperature. Starting material ethyl (E)-3-(5-(benzylthio)-2-nitrophenyl)acrylate (Compound 3a, 1.0 eq, 34.0 kg) was added in portions during period of 3 hours, while maintaining the internal temperature at about 70° C. to 80° C. The temperature rise was slow. The mixture was stirred at 80° C. for 1 hour. LCMS showed the starting material Compound (3a) was consumed and about 93.6% of the product ethyl (E)-3-(5-(benzylthio)-2-nitrophenyl)acrylate (Compound (2a), 3.762 minutes) was formed. The reaction mixture was then cooled down until the internal temperature reached 30° C. Table 15 shows the reaction provided 82% and 82.5% yield of compound (2a) at high purity.

TABLE 15
Optimization condition with 1.1 kg, 20 kg,
and 67.5 kg scale starting material (3a):

| | | | Condition | | | | | |
|---|---|---|---|---|---|---|---|---|
| | | | Fe | NH₄Cl | | Time | | |
| No | (3a) | | (eq) | (eq) | T(° C.) | (h) | EtOH, H₂O | (2a) |
| 1 | 800 | g | 3.0 | 5.0 | 70-80 | 1 | 4 V, 2 V | 630 g |
| | | | | | | | | (98.3% purity, |
| | | | | | | | | 82.0% yield) |
| 2 | 2.5 | kg (2X) | 3.0 | 5.0 | 70-80 | 1 | 4 V, 2 V | 5.50 kg |
| | 2 | kg | | | | | | (97.2% purity, |
| | | | | | | | | 82.5% yield) |

Work-up procedure: the three reactions performed above were combined for workup. EtOAc (3.0 V, 102 L) was then added and the reaction mixture was then agitated for 30 minutes. The suspension was then filtered through a pad of CELITE® and the filter cake was washed with ethyl acetate (6.0 V, 204 L). Organic phases were combined, washed with water (2.0 V X 2, 190 L X 2), and checked by KF. (KF: 6.60%). Isopropyl acetate was also used as solvent alternative to EtOAc.

Isolation step: Crystallization procedure the solid obtained from the work-up step above was further washed with n-heptane (9.0 V, 857 L). The mixture was then heated until the internal temperature reached 60° C. and stirred at 60° C. for 1 hour. The mixture was then cooled down to 25° C. internal temperature and stirred at 25° C. for 12 hours. The solid was then filtered and the filter cake was washed with n-heptane (2.0 V, 190 L). The solid was then dried under N₂ gas. Yellow solid (81.0 kg, 98.7% purity, 82.5% yield, QNMR 92.6%). Mp: 91° C. ¹H NMR (500 MHZ, chloroform-d) δ 7.69 (d, J=15.7 Hz, 1H), 7.30-7.11 (m, 7H), 6.58 (d, J=8.3 Hz, 1H), 6.22 (d, J=15.8 Hz, 1H), 4.26 (q, J=6.9 Hz, 2H), 3.94 (s, 2H), 1.34 (t, J=6.9 Hz, 3H).

TABLE 16
Various conditions of crystallization step after step (c1) of compound (2a):

| | Crude | | | | | | | | |
|---|---|---|---|---|---|---|---|---|---|
| | Product | | Solvent | Volume | Temperature | Product | | Purity | yield |
| 1 | 10.0 | g | MTBE | 3 v | 55° C. to 0° C. | 5.20 | g | 97.3% | 55.2% |
| 2 | 2.00 | g | IPAC | 4 v | 60° C. to 23° C. | 1.00 | g | 98.6% | 53.8% |
| 3 | 0.3 | g | n-Heptane:IPAC = 1:1 | 4 v | 60° C. to 23° C. | 0.23 | g | 99.8% | 83.6% |
| 4 | 0.3 | g | n-Heptane:IPAC = 2:1 | 6 v | 60° C. to 23° C. | 0.21 | g | 99.0% | 75.7% |
| 5 | 0.3 | g | n-Heptane:IPAC = 3:1 | 6 v | 60° C. to 23° C. | 0.25 | g | 97.3% | 88.6% |
| 6 | 21.0 | g | n-Heptane:IPAC = 3:1 | 6 v | 60° C. to 23° C. | 18.0 | g | 98.8% | 92.5% |
| 7 | 160 | g | n-Heptane:IPAC = 3:1 | 6 v | 60° C. to 23° C. | 135 | g | 98.3% | 92.4% |
| 8 | 15.0 | g | n-Heptane:IPAC = 3:1 | 6 v | 60° C. to 23° C. | 12.9 | g | 97.4% | 91.5% |
| 9 | 15.0 | g | n-Heptane:IPAC = 3:1 | 6 v | 60° C. to 23° C. | 12.4 | g | 99.2% | 89.6% |

### Example 3b

Step (c2): Preparation of ethyl (E)-3-(5-(benzylthio)-2-nitrophenyl)acrylate (Compound 2a) via Enzymatic Reduction

Preparation of Tricine Buffer Solution: 330 mmol of Tricine buffer solution was prepared using 54 g Tricine and 900 mL water. The pH was adjusted with 10 N NaOH to pH=8.0.

Preparation of Compound (2a) and (2a-HCl Salt):

To a 500 ml flask was added the starting material Compound (3a), 25.00 g, 66.08 mmol, 95 mass %) and DMSO (7.5 mL/g, 2640 mmol, 100 mass %). The mixture was stirred for 30 mins to dissolve all solid.

To a separate 1-L reactor was added ammonium metavanadate (1.00 equiv., 66.08 mmol, 100 mass %), NR-55 Enzyme (0.10 g/g, 100 mass %), dextrose (4.5 equiv., 297.4 mmol, 100 mass %), GDH-101 (0.025 g/g, 100 mass %), beta-nicotinamide adenine dinucleotide phosphate disodium salt (0.040 g/g, 1.245 mmol, 98 mass %), and 440 mL of the above Tricine buffer, (17.5 vol, pH =8.0, 330 mmol). The reactor was heated to about between 40° C. to 45° C. and the DMSO solution of the substrate was then slowly added over a 1.5-hour period by using a syringe pump. Once the addition was complete, the pH of the reaction was checked and was found to be pH=6.5. The pH was then adjusted to 8.0 by using 10 N NaOH. Sample was taken for IPC. Overnight 88% conversion.

Additional beta-nicotinamide adenine dinucleotide phosphate disodium salt (0.01 g/g, 0.3112 mmol, 98 mass %), and GDH-101 (0.01 g/g, 100 mass %) were then added to the reaction mixture, and stirring was continued for an additional 48 hours. The reaction mixture was then filtered to produce a crude yellow solid.

Work-up and isolation procedure: The solution was filtered and washed with water. The crude solid was slurried at 45° C. with 15 volume with methyl THF overnight. The slurry was then filtered through CELITE and concentrated to an oil. The oil was then diluted with 20 volume Methyl THF and HCl in CPME (1.5 equiv., 99.12 mmol, 3 mol/L) was added at room temperature. The mixture was then stirred for 2 h and then filtered and washed with methyl THF, then with MTBE. 21.1 g of compound (2a HCl salt) was isolated. 100 wt %. 99.28 AP. 91.5% yield. Mp: 155° C. ¹H NMR (500 MHZ, DMSO-d) δ 9.41 (s, 2H), 7.83 (d, J=15.7 Hz, 1H), 7.70 (s, 1H), 7.36-7.20 (m, 7H), 6.61 (d, J=15.7 Hz, 1H), (m, 5H), 4.27 (s, 1H), 4.20 (q, J=7.1 Hz, 2H), (s, 1H), 1.27 (t, J=7.1 Hz, 3H).

### Example 4

Steps (a)+(b): Preparation of ethyl (E)-3-(5-(benzylthio)-2-iodophenyl)acrylate (Compound 1a)

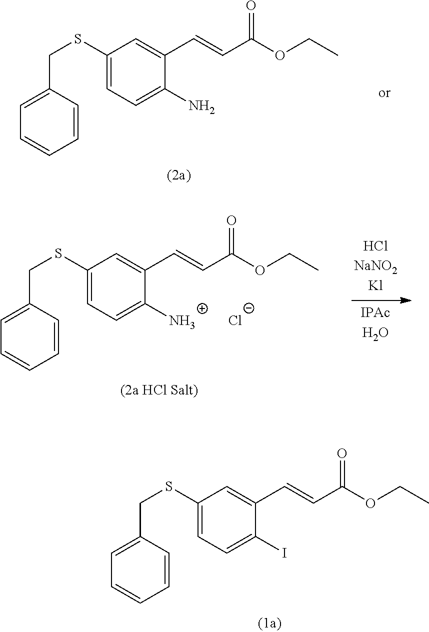 [SMILES: CCOC(=O)/C=C/c1cc(SCc2ccccc2)ccc1N ; CCOC(=O)/C=C/c1cc(SCc2ccccc2)ccc1[NH3+] ; [Cl-] ; CCOC(=O)/C=C/c1cc(SCc2ccccc2)ccc1I]

Reaction procedure: An inert vessel A was purged under nitrogen gas three times The reactor jacket temperature was set to 25±5° C. Isopropyl acetate (15.0 V, 825 L) was charged to vessel A. Starting material ethyl (E)-3-(5-(benzylthio)-2-nitrophenyl)acrylate (Compound (2a), 1.0 eq, 55.0 kg) was then added, followed by HCl (1.5 M, 5.0 eq, 585 L). The mixture was then agitated for 20 minutes, and it was observed that the reaction fluid changed from clear yellow to yellow turbid. The mixture was then cooled down to reach between 0° C. and 5° C. internal temperature. NaNO₂ (2.0 eq, 24.2 kg) in H₂O (1.0 V, 55.0 L) was slowly added at between 0° C. and 5° C. internal temperature, while the internal temperature rose slowly (exotherm). The mixture was stirred at between 0° C. and 5° C. for 2 hours. HPLC showed the starting material Compound (2a) was consumed.

KI (2.5 eq, 72.6 kg) in water (1.0 V, 55.0 L) was then slowly added into the mixture while maintaining the temperature between 0° C. to 5° C. (slight exotherm) and the reaction fluid was observed to change from yellow turbid to brown clear. The reaction mixture was then stirred at the internal temperature between 0° C. and 5° C. for 5 hours. The second HPLC showed about 89.8% of the product compound (1a) was formed at 4.368 min. K₃PO₄ (50.0%, 5.0 V, 275 L) solution in water was then added to quench the reaction mixture at the internal temperature between 0° C. and 5° to achieve an apparent pH=10. (Slow rise in temperature).

In an alternative separate reaction, compound (2a HCl salt) product of Example 3b was used as starting material for Example 4 and under the same reaction conditions for 2a.

TABLE 17
Optimization condition with variou scale starting material (2a) to form (1a):

| | Cmp. | | Condition | | | | |
|---|---|---|---|---|---|---|---|
| Entry | (2a) (g) | | LiCl/DIEA (eq.) | T(° C.) | Time (h) | IPAC | (1a) (g) |
| 1 | 100 | g | NaNO₂ (2.0 eq), | 0 to 5 | 7 | 15 V | 96.0 g |
| | | | KI (2.5 eq), | | | | (100% purity) |
| | | | 1.5M HCl (5.0 eq) | | | | |
| 2 | 630 | g | NaNO₂ (2.0 eq), | 0 to 5 | 7 | 15 V | 592 g |
| | | | KI (2.5 eq), | | | | (98.5% purity, |
| | | | 1.5M HCl (5.0 eq) | | | | 69.4% yield) |
| 3 | 25 | g | NaNO₂ (2.0 eq), | 0 to 5 | 7 | 15 V | 96.0 g |
| | | | KI (2.5 eq), | | | | (100% purity) |
| | | | 1.5M HCl (5.0 eq) | | | | |
| 4 | 13 | kg | NaNO₂ (2.0 eq), | 0 to 5 | 7 | 15 V | 56.0 kg |
| | | | KI (2.5 eg), | | | | (99.0% purity, |
| | | | 1.5M HCl (5.0 eq) | | | | 66.2% yield |
| 5 | 55 | kg | NaNO₂ (2.0 eq), | 0 to 5 | 7 | 15 V | |
| | | | KI (2.5 eq), | | | | |
| | | | 1.5M HCl (5.0 eq) | | | | |

Cmp. means Compound.

TABLE 18
HPLC method for determination of elemental sulfur
generated under various work-up step conditions:

| Entry | Cmp. (2a) (g) | Work-up Condition | IPC (HPLC) |
|---|---|---|---|
| 1 | 100 g | Na₂S₂O₃ q. | HPLC did not detect |
| | | | any sulfur. |
| 2 | 630 g | Na₂S₂O₃ aq. | HPLC detected at 8.262 |
| | | | mins (4680 ppm) |
| 3 | 100 g | Na₂S₂O₃ aq. | HPLC did not detect |
| | | | any sulfur. |
| 4 | 100 g | Sodium | HPLC did not detect |
| | | ascorbate aq. | any sulfur. |
| 5 | 1 kg | NaHSO₃ aq. | HPLC did not detect |
| | | | any sulfur. |

Work-up procedure: The 55.0 kg and 13.0 kg of (2a) scale preparations were combined for workup.

The reaction was quenched with sodium bisulfite (2.5 eq, 3.0 V, 165 L). (note: the use of Na-bisulfite rather than Na-thiosulfate is critical in preventing the formation of elemental sulfur in the work up). The mixture was then heated to reach 25° C. internal temperature and stirred at 25° C. for 30 minutes. The layers were split and the organic phase was washed with water (5 V, 275 L).

Distillation procedure: The organic phase was distilled down to about 3 V EtOAc. The mixture was then heated until the internal temperature reached 50° C. to 55° C. and methanol (12 V, 816 L) was added. The mixture was then agitated for 30 minutes and cooled down to 25° C. internal temperature. The mixture was then agitated at 25° C. for 3 hours. The mixture was then cooled down to reach 0° C. internal temperature and agitated for 12 hours.

Isolation procedure: The mixture was filtered and the solid obtained from the distillation step above was filtered and the filter cake was further washed with MeOH (2.0 V, 136 L). The solid was then dried under N₂ gas. Brown solid; (56.0 k g; 99.0% purity; 66.2% yield). Mp: 79° C. ¹H NMR (500 MHz, chloroform-d) δ 7.79 (d, J=15.8 Hz, 1H), 7.73 (d, J=8.2 Hz, 1H), 7.39 (d, J=2.2 Hz, 1H), 7.32-7.23 (m, 5H), 6.96 (dd, J=8.2, 2.3 Hz, 1H), 6.17 (d, J=15.7 Hz, 1H), 4.28 (q, J=7.1 Hz, 2H), 4.10 (s, 1H), 1.35 (t, J=7.1 Hz, 3H).

Purification step: Crude product (89.0% purity). Crude product was re-slurried in a mixture of MeOH/EtOAc (4:1) 10 vol by heating to 58° C., cooling to 0° C. and filtering the solid product at 0° C.

TABLE 19
Various reaction condition testing the solvent in purification step after steps (a) + (b):

| | Crude | | | | | | | | | |
|---|---|---|---|---|---|---|---|---|---|---|
| | product | | Solvent | Volume | | Temperature | Product | | Purity | Yield |
| 1 | 0.5 | g | MTBE | 2 | v | 60° C. to 23° C. | 0.34 | g | 95.4% | 72.8% |
| 2 | 0.5 | g | Isopropyl ether | 3 | v | 60° C. to 23° C. | 0.38 | g | 91.5% | 78.1% |
| 3 | 0.5 | g | Heptane:EtOAc = 1:1 | 2 | v | 60° C. to 23° C. | 0.34 | g | 94.5% | 72.2% |
| 4 | 0.5 | g | Heptane:EtOAc = 2:1 | 3 | v | 60° C. to 23° C. | 0.32 | g | 93.9% | 67.5% |
| 5 | 3.00 | g | MeOH:EtOAc = 4:1 | 10 | v | 58° C. to 0° C. | 2.32 | g | 99.4% | 86.3% |
| 6 | 15.0 | g | MeOH:EtOAc = 4:1 | 10 | v | 58° C. to 0° C. | 11.2 | g | 99.5% | 83.9% |

The foregoing invention has been described in some detail by way of illustrations and examples, for purposes of clarity and understanding. Those skilled in the art understand that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited herein are hereby incorporated by reference in their entirety for all purposes to the same extent as if each individual patent, patent application or publication were so individually denoted.

We Claim:

1. A process for the preparation of a compound having formula (1), or a pharmaceutically acceptable salt thereof: 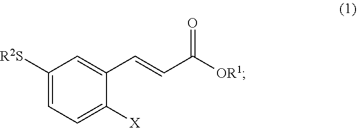
wherein:
X is halo, CN, CF₃, or OH;
R¹ is (C₁-C₆)alkyl; a 5-, 6-, 7-, 8-, 9-, or 10-membered aryl or heteroaryl; or a 3-, 4-, 5-, 6-, 7-, 8-, 9-, or 10-membered cycloalkyl or heterocycloalkyl group;
wherein the heteroaryl or heterocycloalkyl group can have from 1 to 3 heteroatoms independently selected from O, N or S;
or a carbon atom in the cycloalkyl or heterocycloalkyl group can be part of a C═O group;
R² is selected from (C₁-C₆) alkyl or benzyl;
comprising:
(a) contacting a compound of formula (2): 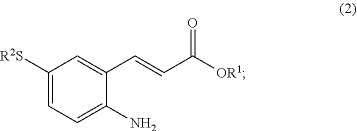
wherein said R¹ and R² are as defined above in compound (1); with an acid HA in a solvent-1, and in the presence of a nitrous salt;
(b) introducing a halogenating agent, a cyanating agent, a trifluoromethylation agent, or a hydroxylating agent, optionally in the presence of a solvent-2, to form said compound (1); wherein said solvent-1 and solvent-2 can be identical or different; and
Wherein said compound (2) is prepared in a process free of palladium catalyst.

2. The process according to claim 1, further comprising preparing said compound (2) comprising:
(c1) activating a metal in the presence of an acidic salt HA¹ in a polar solvent; and contacting a compound of formula (3): 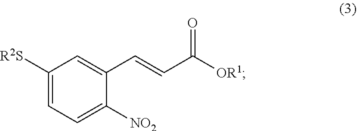
wherein said R¹ and R² are as defined above in compound (1);
with said activated metal at an elevated temperature in a polar solvent to form said compound (2) or a salt thereof:
or alternatively, the process further comprising preparing said compound (2) comprising:
(c2) reacting said compound of formula (3), optionally in the presence of a co-solvent, with an enzymatic reducing agent in an aqueous buffer solution, and in the presence of at least one catalyst and a co-factor, to form said compound of formula (2), or a salt thereof.

3. The process according to claim 2, comprising:
(c1) activating a metal in the presence of an acidic salt HA¹ in a polar solvent; and contacting said compound of formula (3) with said activated metal at an elevated temperature in a polar solvent to form said compound (2) or a salt thereof.

4. The process according to claim 2, comprising:
(c2) contacting said compound of formula (3), in the presence of a co-solvent, with an enzymatic reducing agent in an aqueous buffer, and in the presence of a metal catalyst, a co-catalyst, and a co-factor, to form said compound of formula (2), or a salt thereof.

5. The process according to claim 2, further comprising preparing said compound (3), or a salt thereof, comprising:
(d) contacting a compound of formula (4): 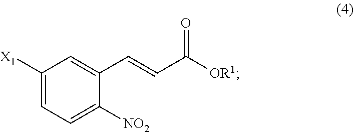
wherein X₁ is halo; and said R¹ is as defined above in compound (3);
with a thiol agent in the presence of a base, in an organic solvent, and at an elevated temperature; to form said compound (3), or a salt thereof.

6. The process according to claim 5, further comprising preparation of said compound (4) comprising:
(e) contacting a compound of formula (5): 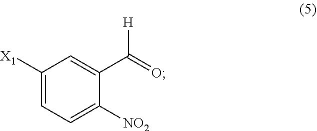
wherein X₁ is halo as defined in compound (4); with an alkenating agent, in the presence of a base, in an organic solvent to form said compound (4), or a salt thereof.

7. The process according to claim 1, wherein in (a), said acid HA is HCl, HBr, HI, p-TsOH, or H₂SO₄.

8. The process according to claim 1, wherein in (a), said solvent-1 is water, THF, methyl THF, CH₃CN, or (C₁-C₆)alkyl acetate solvent, or mixtures thereof.

9. The process according to claim 1, wherein in (b), said halogenating agent is a metal halide salt; said cyanating agent is CuCN; said trifluoromethylation agent is CuCF₃; and said hydroxylating agent is Cu₂O/Cu(II).

10. The process according to claim 1, wherein in (b), said solvent-2 is water, THF, methyl THF, CH₃CN, or (C₁-C₆)alkyl acetate solvent, or mixtures thereof.

11. The process according to claim 2, wherein in (c1), said metal is selected from Fc°, Zn°, Pd°, Pt°, Ru°, or Rh°.

12. The process according to claim 2, wherein in (c1), said acidic salt HA¹ is ammonium chloride, acetic acid, or HCl.

13. The process according to claim 2, wherein in (c1), said polar solvent is water, (C₁-C₆)alkyl alcohol, or mixture thereof.

14. The process according to claim 2, wherein in (c2), said co-solvent is selected from DMSO, or water/DMSO mixture.

15. The process according to claim 2, wherein in (c2), said buffer is selected from phosphate, PIPES, TRICINE, BICINE, HEPES, TRIS, TES, CAPS, Kpi, or CHES.

16. The process according to claim 2, wherein in (c2), said enzymatic reducing agent is a nitroreductase enzyme.

17. The process according to claim 2, wherein in (c2), said catalyst is a metal catalyst and a co-catalyst; wherein said metal catalyst is a vanadium catalyst selected from V₂O₅, NH₃VO₄, V(IV) oxide phthalocyanine, V(IV) oxide bis (2,4-pentanedionate), vanadyl sulfate hydrate, V(V)oxy triethoxide, 3% V/C, or V(III)2,4-pentanedionate.

18. The process according to claim 2, wherein in (c2), said reaction is performed at between pH 7 or pH 8.

19. The process according to claim 2, wherein the product of (c2) is a salt selected from a halide salt, selected from HCl salt or HBr salt, or a sulphonic acid salt, selected from mesylate salt, tosylate salt, or aryl sulphonate salt.

20. The process according to claim 5, wherein in (d), said X₁ in each of Compound (4) and (5) is fluoro or chloro.

21. The process according to claim 5, wherein in (d), said base is a carbonate salt or phosphate salt.

22. The process according to claim 5, wherein in (d), said organic solvent is selected from DMF, DMAc, or NMP.

23. The process according to claim 5, wherein in (d), the reaction is performed under a low water content condition and no excess thiol agent is used.

24. The process according to claim 6, wherein in (e), said X₁ in each of Compound (4) and (5) is F or Cl.

25. The process according to claim 6, wherein in (e), said organic solvent is selected from DIPEA, CH₃CN, TEA, N-methyl morpholine, or mixtures thereof.

26. The process according to claim 1, wherein X in compound (1) is iodo.

27. The process according to claim 1, wherein X₁ in each of compounds (1) and (2) is fluoro or chloro.

28. The process according to claim 1, wherein R¹ in each of compounds (1), (2), (3), and (4) is methoxy or ethoxy.

29. The process according to claim 1, wherein R² in each of compounds (1), (2), and (3) is benzyl.

30. The process according to claim 1, wherein the process is performed in the following order to form said compound of Formula (1):
A) Step (e); Step (d); Step (c1) or (c2); and followed by Steps (a)+(b);
B) Step (d); Step (e); Step (c1) or (c2); and followed by Steps (a)+(b); or
C) Step (d); Step (c1) or (c2); Step (e); and followed by Steps (a)+(b).

31. A compound, which is: 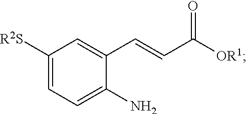
wherein R¹ is ethyl; and R² is benzyl; or a salt thereof.
